(19) United States
(12) Patent Application Publication
(10) Pub. No.: US 2022/0395595 A1
(43) Pub. Date: Dec. 15, 2022

(54) ULTRAVIOLET STERILIZATION APPARATUS AND METHOD OF CONTROLLING THE SAME

(71) Applicants: HYUNDAI MOTOR COMPANY, Seoul (KR); KIA CORPORATION, Seoul (KR)

(72) Inventor: Jae Seung Lee, Hwaseong-si (KR)

(73) Assignees: HYUNDAI MOTOR COMPANY, Seoul (KR); KIA CORPORATION, Seoul (KR)

(21) Appl. No.: 17/549,556

(22) Filed: Dec. 13, 2021

(30) Foreign Application Priority Data
Jun. 9, 2021 (KR) 10-2021-0074562

(51) Int. Cl. A61L 2/10; A61L 2/24; A61L 2/26

(52) U.S. Cl. CPC A61L 2/10; A61L 2/24; A61L 2/26; A61L 2202/11; A61L 2202/16; A61L 2202/14; A61L 2202/25

(57) ABSTRACT

The present disclosure provides an ultraviolet (UV) sterilization apparatus that is capable of changing an emission mode and/or an emission angle. The UV sterilization apparatus includes a sterilization unit configured to be movable to a predetermined position and including a UV emitter, and a driving unit configured to move the sterilization unit.

### CROSS-REFERENCE TO RELATED APPLICATION

This application claims under 35 U.S.C. § 119(a) the benefit of priority from Korean Patent Application No. 10-2021-0074562, filed on Jun. 9, 2021, the entire contents of which are incorporated herein by reference.

### BACKGROUND

### (a) Technical Field

The present disclosure relates to an ultraviolet (UV) sterilization apparatus and, more particularly, to a UV sterilization apparatus capable of changing switching between emission modes and/or emission angles.

### (b) Background Art

Ultraviolet (UV) light is used for a sterilization apparatus because it can promote chemical reactions, oxidize organic matter, and eradicate microorganisms. Recently, in particular, interest in hygiene and sterilization has increased more than ever before due to the coronavirus pandemic.

UV light includes UV-C light having a wavelength in the range of 200 to 280 nanometers (nm), UV-B light having a wavelength in the range of 280 to 315 nm, and UV-A light having a wavelength in the range of 315 to 400 nm. It is known that, when UV-C light, which has a sterilization function, is radiated onto deoxyribonucleic acid (DNA), it destroys the DNA and suppresses the regeneration and replication thereof, thus eradicating viruses. Because it has recently been reported that UV-C light is effective against coronavirus, UV-C light-emitting diode (LED) products for eradicating the coronavirus have been developed.

Due to this trend, attempts are being made to apply a UV sterilization apparatus to a vehicle in order to sterilize the passenger compartment of a vehicle, which is an enclosed space.

The above information disclosed in this Background section is only for enhancement of understanding of the background of the disclosure, and therefore it may contain information that does not form the related art that is already known in this country to a person of ordinary skill in the art.

### SUMMARY OF THE DISCLOSURE

The present disclosure has been made in an effort to solve the above-described problems associated with the related art, and it is an object of the present disclosure to provide a UV sterilization apparatus having improved sterilization performance and reliability.

It is another object of the present disclosure to provide a UV sterilization apparatus capable of switching between emission modes and/or emission angles.

However, the objects to be accomplished by the present disclosure are not limited to the above-mentioned objects, and other objects not mentioned herein may be clearly understood by those having ordinary skill in the art from the following description.

In one aspect, the present disclosure provides an ultraviolet (UV) sterilization apparatus including a sterilization unit configured to be movable to a predetermined position and including a UV emitter. The UV sterilization unit also includes a driving unit configured to move the sterilization unit.

In another aspect, the present disclosure provides a method of controlling a UV sterilization apparatus. The method includes sensing the temperature in an enclosed space in which a sterilization unit is located, driving a motor, configured to move the sterilization unit into or out of an enclosure located in the enclosed space, to move the sterilization unit out of the enclosure located in the enclosed space when the temperature exceeds a predetermined threshold temperature, and driving the motor to move the sterilization unit back into the enclosure located in the enclosed space when the temperature becomes equal to or lower than the threshold temperature.

Other aspects and embodiments of the present disclosure are discussed below.

The above and other features of the disclosure are discussed below.

### BRIEF DESCRIPTION OF THE DRAWINGS

The above and other features of the present disclosure are described in detail below with reference to certain embodiments thereof, illustrated in the accompanying drawings, which are given hereinbelow by way of illustration only, and thus are not limitative of the present disclosure, and wherein:

FIG. 1 illustrates a UV sterilization apparatus according to an embodiment of the present disclosure;

FIG. 2 illustrates an installation example of the UV sterilization apparatus according to an embodiment of the present disclosure;

FIG. 3A illustrates the state in which the UV sterilization apparatus according to an embodiment of the present disclosure is installed in an overhead console of a vehicle;

FIG. 3B illustrates the state in which the position of the UV sterilization apparatus shown in FIG. 3A is changed;

FIG. 4 illustrates a UV sterilization apparatus according to an embodiment of the present disclosure;

FIG. 5 is a bottom view of FIG. 4;

FIG. 6 is a front view of the portion indicated by the dotted line in FIG. 4;

FIG. 7 illustrates the UV sterilization apparatus according to an embodiment of the present disclosure;

FIGS. 8 and 9 are cross-sectional views taken along line A-A′ in FIG. 7;

FIGS. 10A and 10B are partial enlarged views of FIG. 4;

FIGS. 11 and 12 illustrate a UV sterilization apparatus according to an embodiment of the present disclosure;

FIG. 13 is an enlarged view of the portion indicated by the dotted line in FIG. 12;

FIG. 14 illustrates the configuration of the UV sterilization apparatus according to an embodiment of the present disclosure;

FIG. 15 is a control flowchart of the UV sterilization apparatus according to an embodiment of the present disclosure under a high-temperature condition;

FIG. 16A illustrates the operation standby state of a sterilization unit according to an embodiment of the present disclosure in an overhead console;

FIG. 16B illustrates the state in which the sterilization unit according to an embodiment of the present disclosure descends from the overhead console;

FIG. 17 is a control flowchart of the UV sterilization apparatus according to an embodiment of the present disclosure when responding to a user request;

FIG. 18A is a front view of the UV sterilization apparatus according to an embodiment of the present disclosure when the lengths of first and second wires are set to minimum lengths;

FIG. 18B is a front view of the UV sterilization apparatus according to an embodiment of the present disclosure when the lengths of the first and second wires are adjusted to the maximum lengths;

FIG. 19 is a control flowchart of a UV sterilization apparatus according to an embodiment of the present disclosure;

FIGS. 20A to 20C illustrate the UV sterilization apparatus that is controlled according to the control flowchart shown in FIG. 19; and

FIGS. 21 and 22 illustrate the characteristics of a UV-C LED, specifically a change in the performance of a UV emitter depending on a change in temperature.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes, will be determined in part by the particular intended application and use environment.

In the figures, reference numbers refer to the same or equivalent parts of the present disclosure throughout the several figures of the drawing.

### DETAILED DESCRIPTION

Hereinafter, embodiments of the present disclosure are described in detail with reference to the accompanying drawings. Specific structures or functions described in the embodiments of the present disclosure are merely for illustrative purposes. Embodiments according to the concept of the present disclosure may be implemented in various forms, and it should be understood that they should not be construed as being limited to the embodiments described in the present specification, but include all of modifications, equivalents, or substitutes included in the spirit and scope of the present disclosure.

It is understood that, although the terms “first,” “second,” and the like may be used herein to describe various elements, these elements should not be limited by these terms. These terms are only used to distinguish one element from another element. For instance, a first element discussed below could be termed a second element without departing from the teachings of the present disclosure. Similarly, the second element could also be termed the first element.

It is understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present. Other expressions that explain the relationship between elements, such as “between,” “directly between,” “adjacent to,” or “directly adjacent to,” should be construed in the same way.

Like reference numerals denote like components throughout the specification. In the meantime, the terminology used herein is for the purpose of describing particular embodiments only and is not intended to be limiting. As used herein, the singular forms “a,” “an,” and “the” are intended to include the plural forms as well, unless the context clearly indicates otherwise. It is further understood that the terms “comprise,” “include,” “have,” and the like, when used in this specification, specify the presence of stated components, steps, operations, and/or elements, but do not preclude the presence or addition of one or more other components, steps, operations, and/or elements thereof.

An object of the present disclosure is to provide a UV sterilization apparatus for sterilizing an enclosed indoor space, particularly the passenger compartment of a vehicle.

With regard to a UV-C LED, as the distance from an emission source to a target to be sterilized increases, or depending on an emission angle, the intensity of light decreases, and the sterilization performance is degraded. Therefore, the longer the distance between the UV-C LED and the target to be sterilized, the longer the emission time.

Further, a UV-C LED is less effective and is more prone to failure in a high-temperature environment. When a UV-C LED operates in a high-temperature environment, for example, such as when a vehicle is parked under direct sunlight, the effect of the LED may be degraded, or may break down due to high temperatures.

In general, in the passenger compartment of a vehicle, large amounts of bacteria are present on a steering wheel provided in front of a driver's seat, door handles, seat belts, cup holders, a gear selector, and a center fascia. Therefore, the sterilization apparatus needs to be installed in a vehicle so that the sterilization effect is applied to various points in the passenger compartment of the vehicle.

An object of the present disclosure is to provide a UV sterilization apparatus and a method of controlling the same capable of preventing degradation of sterilization performance due to an increase in the distance to a target to be sterilized, of preventing deterioration in the performance of the sterilization apparatus in a high-temperature environment, and of providing a sterilization effect to various points in the passenger compartment of a vehicle.

Hereinafter, the present disclosure is described in detail with reference to the accompanying drawings.

As shown in FIG. 1, a UV sterilization apparatus 1 according to the present disclosure may be used in any indoor space requiring sterilization. In particular, the UV sterilization apparatus according to the present disclosure may achieve sterilization more effectively when used in a confined indoor space, for example, the passenger compartment of a vehicle.

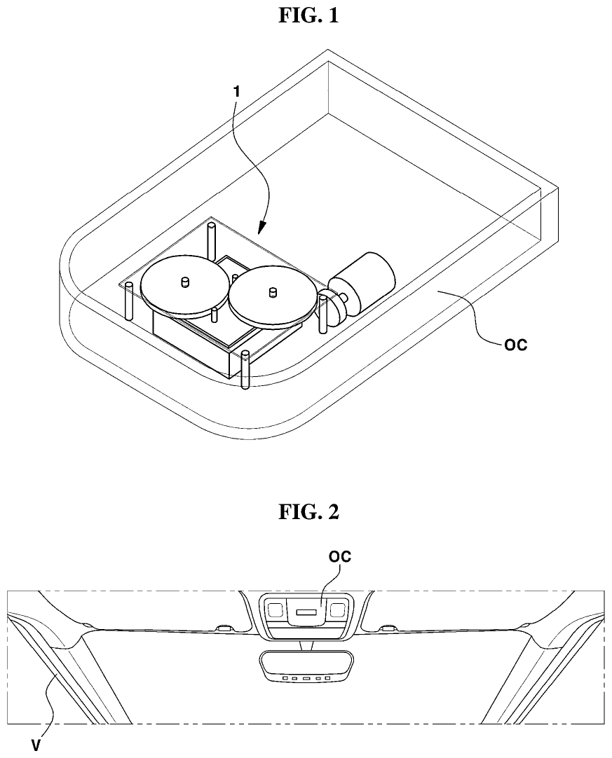

The UV sterilization apparatus 1 according to the present disclosure may be mounted at any position in a vehicle V. In particular, as shown in FIG. 2, the UV sterilization apparatus 1 may be mounted to an upper side in the passenger compartment of the vehicle V, for example, the roof, the head lining, or the overhead console OC of the vehicle V. Hereinafter, the case in which the UV sterilization apparatus 1 is mounted in the overhead console OC is described by way of example. However, the UV sterilization apparatus 1 is not necessarily mounted in the overhead console OC, and may be mounted at any position, so long as the apparatus 1 is capable of vertically moving.

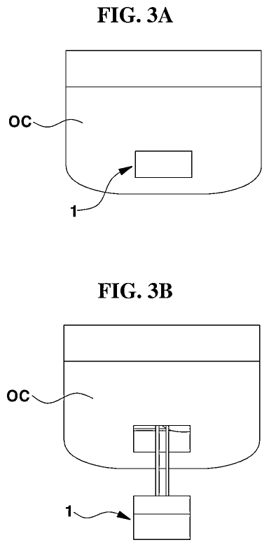

As shown in FIGS. 3A and 3B, the UV sterilization apparatus 1 according to the present disclosure is configured to be movable. The UV sterilization apparatus 1 is configured to be capable of ascending and descending in the state of being mounted in the overhead console OC. In particular, the UV sterilization apparatus 1 is formed to be movable upwards and downwards in order to satisfy temperature conditions and/or improve sterilization performance. This is described below.

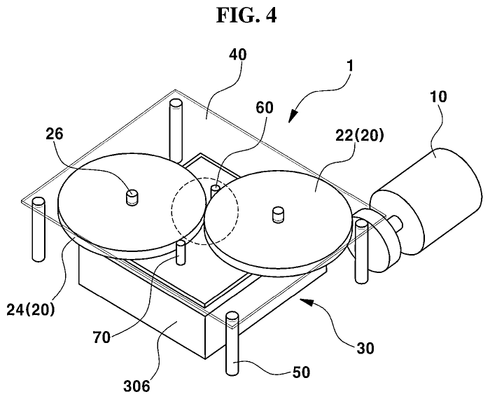

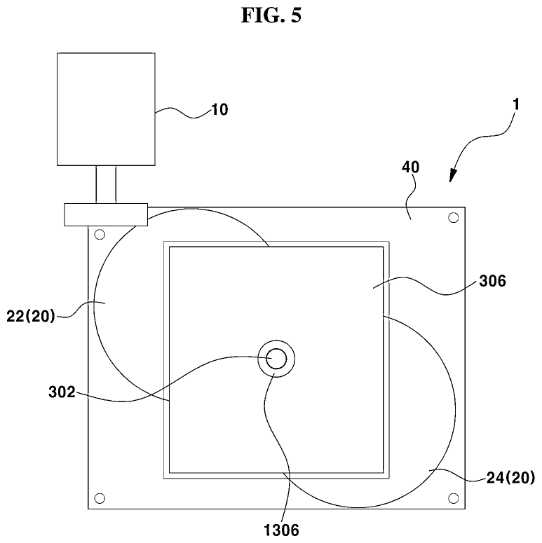

As shown in FIGS. 4 and 5, according to an embodiment of the present disclosure, the UV sterilization apparatus 1 includes a motor 10, a pulley 20, and a sterilization unit 30 to ascend and descend. The motor 10 and the pulley 20 are fixed, and the sterilization unit 30 is configured to be moved upwards and downwards by the operation of the motor 10 and the pulley 20.

The motor 10 and the pulley 20 are fixed in the overhead console OC, and the motor 10 provides driving force to the pulley 20. According to an embodiment of the present disclosure, the pulley 20 includes a first pulley 22 and a second pulley 24.

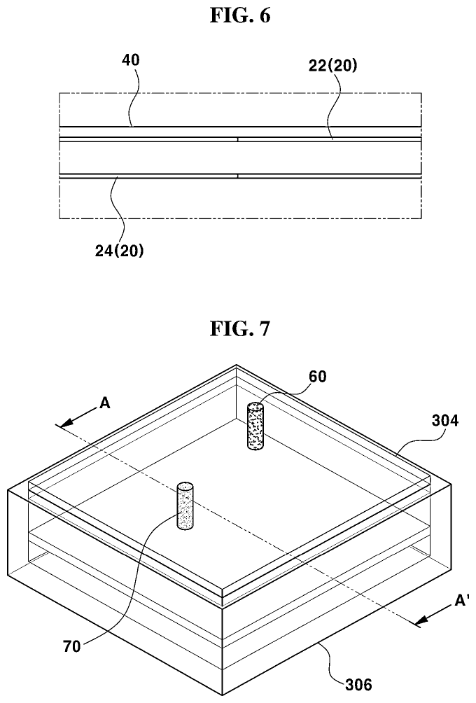

The pulley 20 may be fixed in the overhead console OC through a fixing panel 40. The fixing panel 40 is coupled to the overhead console OC through a support pin 50. Each of the first pulley 22 and the second pulley 24 is rotatably mounted to the fixing panel 40 through a pulley pin 26. In addition, the pulley 20 is configured such that, when the first pulley 22 is rotated, the second pulley 24 is also rotated together therewith. More specifically, referring to FIG. 6, when the motor 10 provides rotational force to the first pulley 22, the second pulley 24, which is engaged with the first pulley 22, is also rotated together therewith. In other words, since the first pulley 22 and the second pulley 24 are engaged with each other to rotate simultaneously, it is possible to rotate both the first pulley 22 and the second pulley 24 using a single motor 10.

A first wire 60 is wound around the first pulley 22. When the motor 10 rotates in a first direction to rotate the first pulley 22, the first wire 60 wound around the first pulley 22 may be unwound therefrom. Similarly, a second wire 70 is wound around the second pulley 24. As the second pulley 24 is rotated in association with the first pulley 22, the second wire 70 wound around the second pulley 24 is unwound therefrom. The first wire 60 may be one of an anode wire and a cathode wire, and the second wire 70 may be the other one of the anode wire and the cathode wire.

In addition, when the motor 10 rotates in a second direction, which is opposite the first direction, to rotate the first pulley 22, the unwound first wire 60 may be wound around the first pulley 22 again. Similarly, as the second pulley 24 is rotated in association with the first pulley 22, the unwound second wire 70 may be wound around the second pulley 24 again.

The first wire 60 and the second wire 70 are connected to the sterilization unit 30 to supply power to the sterilization unit 30. The first wire 60 and the second wire 70 are respectively unwound from or wound around the first pulley 22 and the second pulley 24 by the operation of the motor 10 and the first and second pulleys 22 and 24, thereby making it possible to change the position of the sterilization unit 30 with respect to the overhead console OC.

The sterilization unit 30 includes a UV emitter 302, configured to generate a UV ray to perform sterilization. According to an embodiment of the present disclosure, the UV emitter 302 is a light-emitting diode (LED) configured to generate UV radiation, such as, for example, UV-C radiation.

Referring to FIGS. 7-9, the sterilization unit 30 includes an inner cover 304. The inner cover 304 is disposed to face the pulley 20. In particular, the first wire 60 and the second wire 70 may be coupled to the inner cover 304. Accordingly, the sterilization unit 30 may ascend or descend due to a change in the length of the first wire 60 and the second wire 70. The inner cover 304 may be made of an aluminum material, which has excellent heat dissipation performance, but an embodiment is not limited thereto.

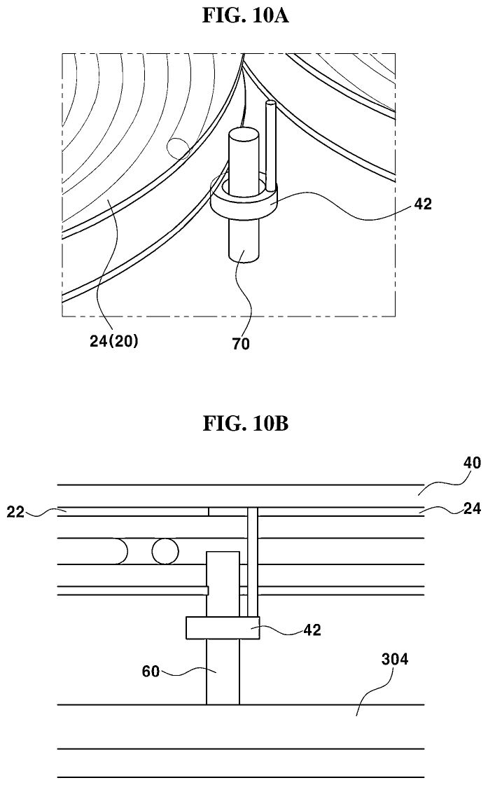

Referring to FIGS. 10A and 10B, according to an embodiment of the present disclosure, at least two wire-fixing rings 42 may be mounted to the fixing panel 40. One of the wire-fixing rings 42 serves to guide the first wire 60 extending from the first pulley 22. Similarly, another one of the wire-fixing ring 42 guides the second wire 70 extending from the second pulley 24.

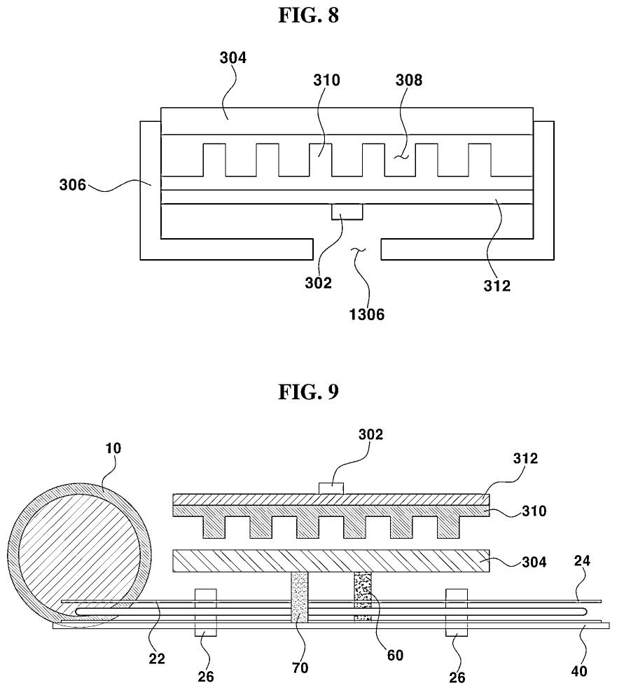

Referring again to FIG. 8, the inner cover 304 is coupled to an outer cover 306, and a space 308 is formed between the inner cover 304 and the outer cover 306. The outer cover 306 accommodates the components of the sterilization unit 30, and an opening 1306 is formed in the bottom or in the center of the lower surface of the outer cover 306. The light generated by the UV emitter 302, which is accommodated in the outer cover 306, is emitted to the outside of the sterilization unit 30 through the opening 1306.

A printed circuit board 312 including a heat sink 310 for heat dissipation is disposed in the space 308. The printed circuit board 312 is configured to receive instructions from a controller 80, such as a vehicle control unit, and to control the operation of the UV emitter 302 disposed under the printed circuit board 312.

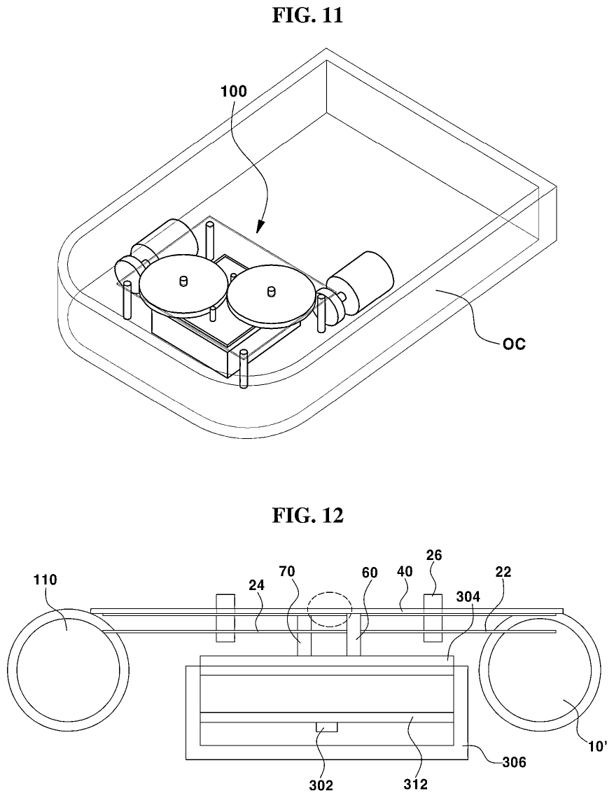

As shown in FIGS. 11 and 12, according to an embodiment of the present disclosure, two motors 10′ and 110 are included in place of a single motor 10. In order to distinguish the same from the UV sterilization apparatus 1 including a single motor 10, the UV sterilization apparatus according to this embodiment is denoted by reference numeral 100. In this embodiment, the two motors are referred to as a first motor 10′ and a second motor 110, respectively.

With this exception, the above description of the UV sterilization apparatus 1 is also applicable to this embodiment, so a duplicate description thereof is omitted, and only differences therebetween are described.

The UV sterilization apparatus 100 according to the present disclosure includes two motors, i.e. the first motor 10′ and the second motor 110. Each of the motors 10′ and 110 is configured to rotate the pulley 20 disposed adjacent thereto. Specifically, the first motor 10′ may rotate the first pulley 22, and the second motor 110 may rotate the second pulley 24.

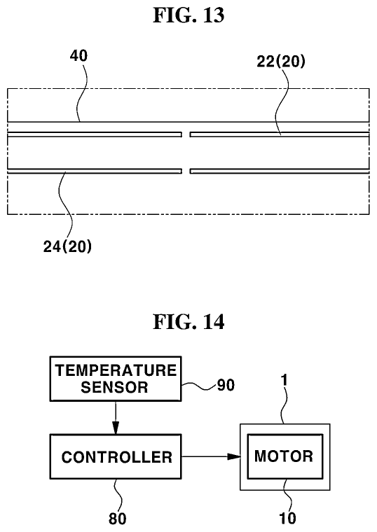

In this case, because the rotation of the first pulley 22 and the rotation of the second pulley 24 are controlled independently of each other, the first pulley 22 and the second pulley 24 are disposed to be spaced a predetermined distance apart from each other, as shown in FIG. 13. In other words, as shown in FIG. 6, when the first pulley 22 and the second pulley 24 are simultaneously operated by a single motor 10, the first pulley 22 and the second pulley 24 are configured to mesh with each other. On the other hand, when the first pulley 22 and the second pulley 24 are respectively operated by the first motor 10′ and the second motor 110, a predetermined spacing distance is set between the first pulley 22 and the second pulley 24.

As shown in FIG. 14, the UV sterilization apparatus 1 according to the present disclosure further includes a controller 80. The controller 80 is configured to move the position of the sterilization unit 30 in response to the input conditions. The input conditions include the temperature in the passenger compartment of a vehicle and/or a request from an occupant of a vehicle. The controller 80 is configured to drive the motor 10 in order to move the position of the sterilization unit 30. In addition, the controller 80 is configured to communicate with a temperature sensor 90, which is configured to measure the temperature in the passenger compartment of a vehicle. Hereinafter, the operation of the controller 80 is described.

The UV sterilization apparatus 1 according to the present disclosure is automatically operated as follows. The UV sterilization apparatus 1 according to the present disclosure may be configured to automatically move in order to prevent deterioration in the performance thereof in a high-temperature environment, i.e., when the temperature in the passenger compartment of a vehicle exceeds a predetermined level.

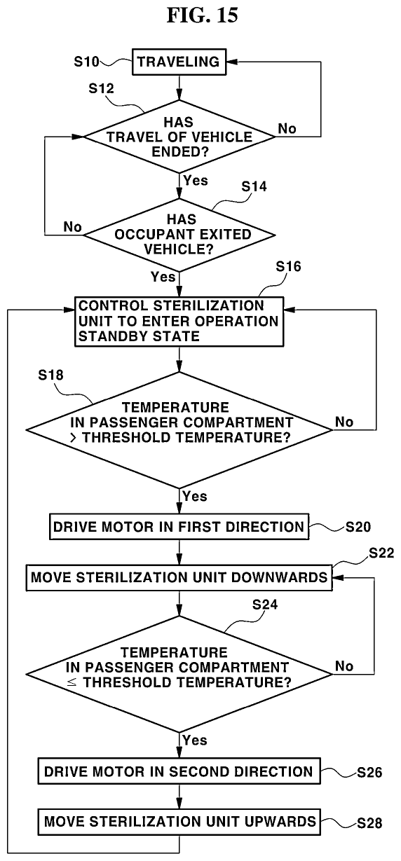

Referring to FIG. 15, the controller 80 determines whether the travel of the vehicle has ended (S10 and S12). For example, the controller 80 may sense whether the travel of the vehicle has ended through communication with the vehicle control unit. At the end of the travel of the vehicle, the sterilization unit 30 is inserted into the overhead console OC. When the occupant gets out of the vehicle, the sterilization unit 30 enters an operation standby state.

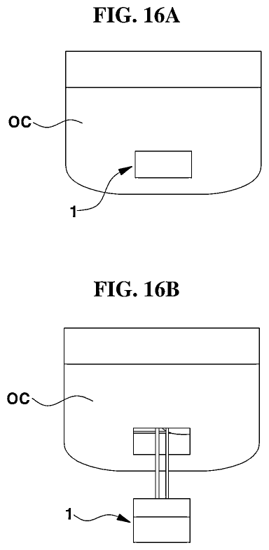

Upon determining that the travel of the vehicle has ended, the controller 80 determines whether there is an occupant in the vehicle (S14). Upon determining that there is no occupant in the vehicle, as shown in FIG. 16A, the sterilization unit 30 enters the operation standby state (S16), at which point the sterilization unit 30 is located in the overhead console OC.

When the sterilization unit 30 is in the operation standby state, the controller 80 receives information about the temperature in the passenger compartment of the vehicle in real time from the temperature sensor 90. For example, when the outdoor temperature is high and the vehicle is parked outdoors, the temperature in the indoor space of the vehicle increases, and the temperature of the sterilization unit 30, which is located in the enclosed space, also increases. Accordingly, upon determining that the temperature in the passenger compartment of the vehicle exceeds a predetermined threshold temperature, for example, 30 degrees Celsius, based on the temperature information received from the temperature sensor 90 (S18), the controller 80 controls the motor 10 to be driven. The controller 80 drives the motor 10 in the first direction so that the sterilization unit 30 descends (S20). The first pulley 22 and the second pulley 24 are rotated by the operation of the motor 10 so that the sterilization unit 30 descends (S22). The sterilization unit 30 descends and is located outside the overhead console OC, as shown in FIG. 16B.

In the state in which the sterilization unit 30 is moved downwards, the controller 80 continuously receives the information about the temperature in the passenger compartment of the vehicle from the temperature sensor 90 and determines whether the temperature in the passenger compartment of the vehicle is lower than or equal to the threshold temperature (S24). Upon determining that the temperature in the passenger compartment of the vehicle is lower than or equal to 30 degrees Celsius, which is the threshold temperature, the controller 80 drives the motor 10 again so that the sterilization unit 30 ascends to the original position thereof. In other words, the controller 80 drives the motor 10 in the second direction, which is opposite the first direction (S26), whereby the sterilization unit 30 ascends to the position shown in FIG. 16A (S28). The present disclosure functions to prevent the sterilization unit 30 from getting hot in the confined space in the overhead console OC in a high-temperature environment and to remove heat from the sterilization unit 30 by moving the same downwards. Accordingly, it is possible to prevent deterioration in the sterilization performance of the sterilization unit 30 due to high temperatures and to improve the durability thereof.

Further, the UV sterilization apparatus 100 including two motors, i.e. the first motor 10′ and the second motor 110, which are independently controlled, may also be operated in a manner similar to that described above. When the temperature in the passenger compartment of the vehicle exceeds the threshold temperature, the controller 80 rotates the first motor 10′ so that the first wire 60 is unwound from the first pulley 22, and rotates the second motor 110 so that the second wire 70 is unwound from the second pulley 24. On the other hand, when the temperature in the passenger compartment of the vehicle drops to the threshold temperature or lower, the controller 80 rotates the first motor 10′ so that the first wire 60 is wound around the first pulley 22, and rotates the second motor 110 so that the second wire 70 is wound around the second pulley 24, thereby moving the sterilization unit 30 upwards.

In addition, according to an embodiment of the present disclosure, the UV sterilization apparatus 1 may be moved when performing sterilization in response to a request from an occupant. The controller 80 is configured to enter the sterilization mode in response to the request from the occupant.

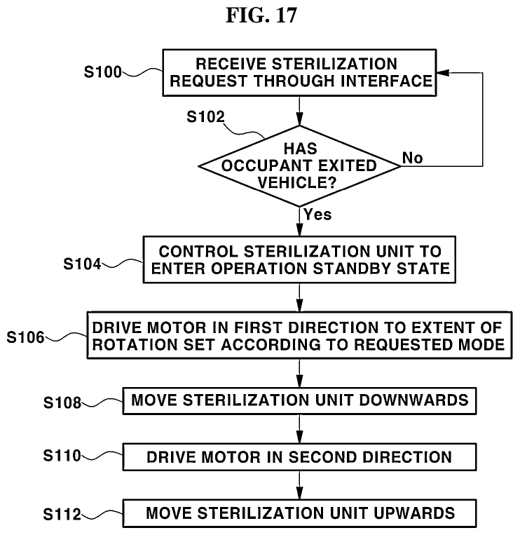

As shown in FIG. 17, the request from the occupant is transmitted to the controller 80 through an interface, such as an audio-video-navigation (AVN) system of the vehicle or a smart device configured to communicate with the vehicle (S100). The request from the occupant may request any of several modes. For example, the requested sterilization mode may include at least a broad-range sterilization mode, an intensive sterilization mode, and an intermediate sterilization mode. The controller 80 may drive the motor 10 according to each mode to adjust the descending lengths of the first wire 60 and the second wire 70. Alternatively, the controller 80 may drive the first motor 10′ and the second motor 110 according to each mode to adjust the descending lengths of the first wire 60 and the second wire 70.

Upon receiving the sterilization request from an occupant, the controller 80 determines whether there is an occupant in the vehicle (S102). Upon determining that there is no occupant in the vehicle, the sterilization unit 30 enters the operation standby state in which the sterilization unit 30 can move (S104).

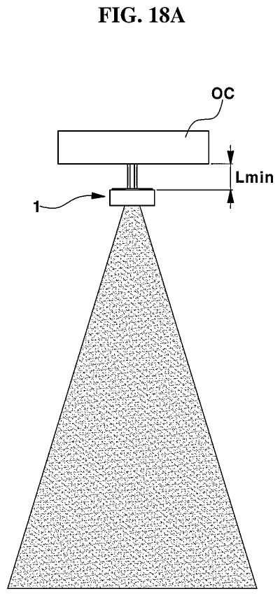

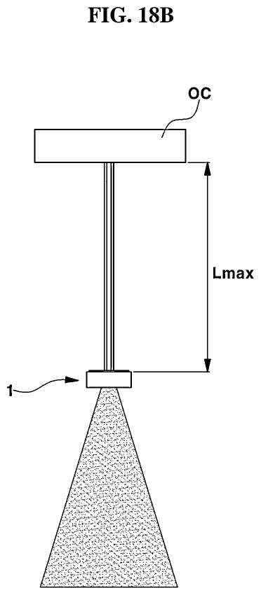

According to each mode, the descending distance of the sterilization unit 30 may be set by adjusting the lengths that the first wire 60 and the second wire 70 are withdrawn out of the overhead console OC. As shown in FIGS. 18A and 18B, according to the requested sterilization mode, the controller 80 controls the withdrawal lengths of the first and second wires 60 and 70 between the minimum lengths Lmin (FIG. 18A) and the maximum lengths Lmax (FIG. 18B) of the first and second wires 60 and 70, thereby enabling sterilization suitable for the requested sterilization mode. In other words, the controller 80 may drive the motor 10 in the first direction to the extent of rotation set according to the requested mode (S106), whereby the sterilization unit 30 descends a predetermined distance (S108).

For example, when the occupant requests broad-range sterilization, the sterilization unit 30 descends a relatively short distance from the overhead console OC (FIG. 18A). On the other hand, when the occupant requests intensive sterilization, the sterilization unit 30 descends the predetermined maximum descending distance from the overhead console OC (FIG. 18B). In addition, when the occupant requests intermediate sterilization, the sterilization unit 30 descends a distance suitable therefor. The sterilization performance of the sterilization apparatus depends on the distance to the target to be sterilized. According to the present disclosure, it is possible to secure excellent sterilization performance by moving the UV sterilization apparatus 1 or 100 close to the target to be sterilized. In addition, the present disclosure provides the broad-range sterilization function and the intensive sterilization function in response to the selection of a user, thereby providing diversity and convenience in sterilization.

After the sterilization unit 30 descends and performs sterilization for a predetermined time, the controller 80 drives the motor 10 in the second direction (S110). Thereby, the sterilization unit 30 ascends and is inserted into the overhead console OC to return to the original position thereof (S112). Alternatively, the controller 80 rotates the first motor 10′ so that the first wire 60 is wound around the first pulley 22, and rotates the second motor 110 so that the second wire 70 is wound around the second pulley 24.

According to the present disclosure, the sterilization unit 30 is configured to be movable to adjust the distance between the sterilization unit and the target to be sterilized, thereby making it possible to improve the sterilization performance.

In addition, according to the present disclosure, when the UV sterilization apparatus 1 is in a high-temperature environment, the sterilization unit 30 is moved out of the overhead console OC to be cooled.

Moreover, according to an embodiment of the present disclosure, the angle of the sterilization unit 30 is changed. In particular, the angle of the sterilization unit 30 may be changed where two motors are independently controlled.

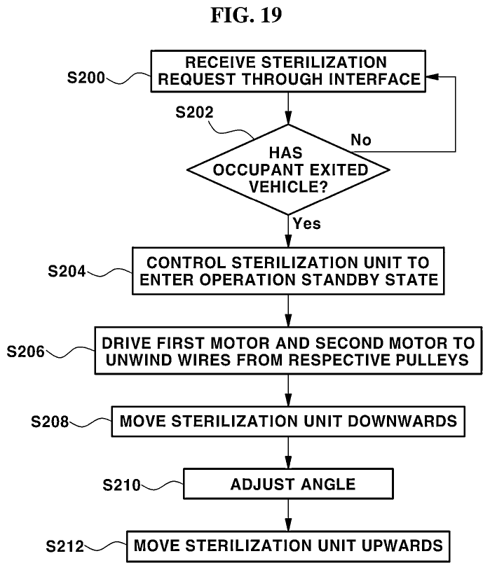

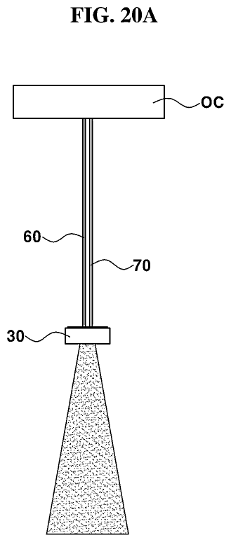

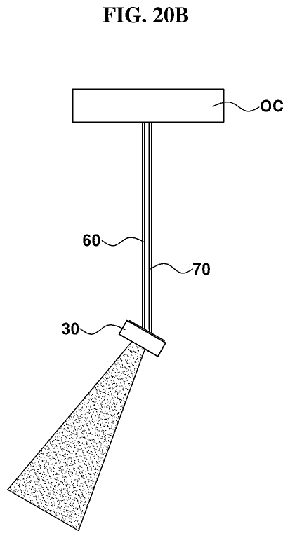

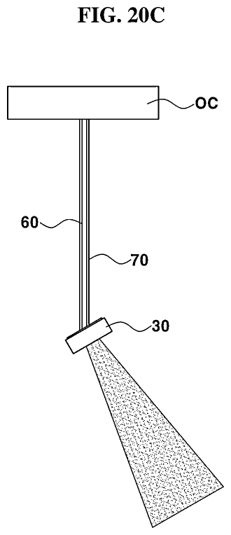

As shown in FIG. 19, the controller 80 receives a sterilization request from an occupant of the vehicle through an interface (S200). After receiving the sterilization request, the controller 80 determines whether the occupant has exited the vehicle (S202). Upon determining that the occupant has exited the vehicle, the controller 80 controls the sterilization unit 30 to enter the operation standby state (S204). The controller 80 drives each of the first motor 10′ and the second motor 110 to the extent of rotation set according to the requested sterilization mode (S206). Accordingly, as shown in FIG. 20A, the first wire 60 and the second wire 70 are respectively unwound from the first pulley 22 and the second pulley 24 by a predetermined length, whereby the sterilization unit 30 descends to a prescribed position (S208).

When the occupant requests intensive sterilization of a specific side of the vehicle, for example, any one of the driver's seat and the front passenger's seat, the controller 80 drives the first motor 10′ and the second motor 110 according to the request. In other words, the controller 80 adjusts the angle of the sterilization unit 30 (S210). For example, when intensive sterilization of the left side is requested, the left wire is wound up a predetermined length so that the sterilization unit 30 is tilted. For example, when the left wire is the first wire 60 and the right wire is the second wire 70, as shown in FIG. 20B, the first wire 60 is wound around the first pulley 22 by a predetermined length, whereby the angle of the sterilization unit 30 is adjusted so that the sterilization unit 30 intensively sterilizes the left side. On the other hand, when intensive sterilization of the right side is requested, the second wire 70 is wound around the second pulley 24 by a predetermined length, whereby the angle of the sterilization unit 30 is adjusted so that the sterilization unit 30 intensively sterilizes the right side (FIG. 20C).

When the sterilization unit 30 finishes sterilization, the controller 80 drives the first motor 10′ and the second motor 110 in order to move the sterilization unit 30 upwards (S212).

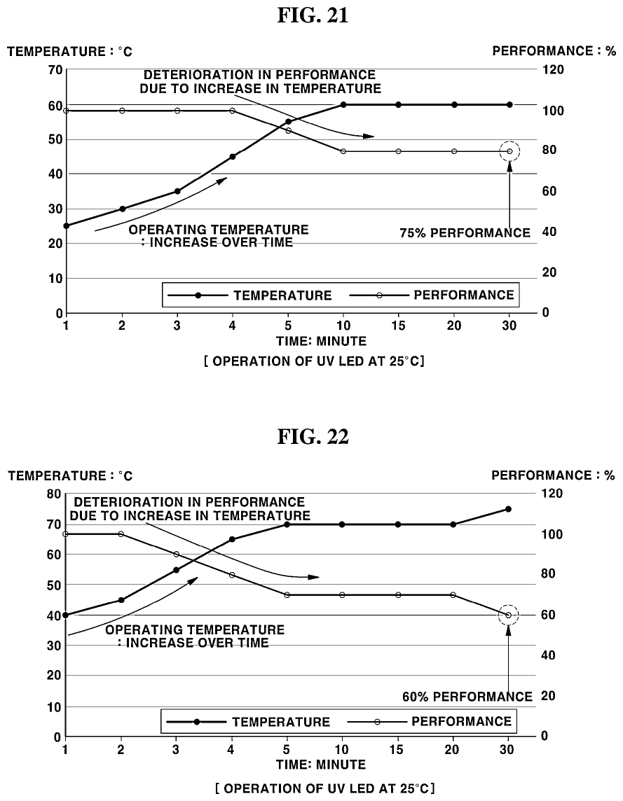

FIGS. 21 and 22 illustrate the characteristics of a UV-C LED, specifically a change in the performance of the UV-C LED depending on a change in temperature. FIG. 21 illustrates a case in which the ambient temperature is 25 degrees Celsius. It can be seen that the operating temperature of the UV-C LED increases over time and that the performance of the UV-C LED decreases by about 25% after a predetermined period of time as the temperature increases.

FIG. 22 illustrates a case in which the ambient temperature is 40 degrees Celsius. It can be seen from FIG. 22 that the performance of the UV-C LED greatly decreases, specifically by about 40%, due to the high temperature after the same operation time period as that shown in FIG. 21.

The temperature of the UV sterilization apparatus changes in proportion to the change in the ambient temperature, and the sterilization performance thereof depends on the temperature thereof. In other words, as the initial temperature of the sterilization unit is set to be lower, it is possible to ensure superior performance thereof and to prevent malfunction thereof due to heat. When the temperature of the sterilization unit is maintained at 30 degrees Celsius or lower, it is possible to guarantee the performance of the UV-C LED up to about 10 minutes, which is the time required for the UV-C LED to perform sterilization. The present disclosure is capable of greatly improving the performance of the UV sterilization apparatus by moving the same to respond to a high-temperature environment.

As is apparent from the above description, the present disclosure provides a UV sterilization apparatus having improved sterilization performance and reliability.

In addition, the present disclosure provides a UV sterilization apparatus capable of variously changing an emission mode and an emission angle.

However, the effects achievable through the present disclosure are not limited to the above-mentioned effects, and other effects not mentioned herein may be clearly understood by those having ordinary skill in the art from the above description.

The disclosure has been described in detail with reference to embodiments thereof. However, it should be appreciated by those having ordinary skill in the art that changes may be made in these embodiments without departing from the principles and spirit of the disclosure, the scope of which is defined in the appended claims and their equivalents.

What is claimed is:

1. An ultraviolet (UV) sterilization apparatus comprising:
a sterilization unit configured to be movable to a predetermined position, the sterilization unit comprising a UV emitter; and
a driving unit configured to move the sterilization unit.

2. The UV sterilization apparatus of claim 1, wherein the driving unit comprises:
a motor configured to provide a rotating force; and
a pulley configured to be rotated by the motor, and
wherein a wire connected to the sterilization unit is wound around or unwound from the pulley.

3. The UV sterilization apparatus of claim 2, wherein the pulley comprises:
a first pulley configured to directly receive a rotating force from the motor; and
a second pulley engaged with the first pulley to be rotated together with the first pulley, and
wherein a first wire is wound around the first pulley, and a second wire is wound around the second pulley.

4. The UV sterilization apparatus of claim 2, wherein the motor comprises a first motor and a second motor,
wherein the first motor is configured to rotate a first pulley around which a first wire is wound, and
wherein the second motor is configured to rotate a second pulley around which a second wire is wound.

5. The UV sterilization apparatus of claim 2, further comprising:
a wire-fixing ring configured to guide the wire unwound from the pulley.

6. The UV sterilization apparatus of claim 2, wherein the sterilization unit comprises:
a cover connected to the wire; and
a printed circuit board accommodated in the cover, and
wherein the UV emitter is mounted on the printed circuit board, and wherein the printed circuit board controls operation of the UV emitter.

7. The UV sterilization apparatus of claim 6, further comprising:
a temperature sensor configured to sense a temperature of the sterilization unit; and
a controller configured to control operation of the motor based on a temperature received from the temperature sensor.

8. The UV sterilization apparatus of claim 7, wherein the controller is configured to receive a sterilization mode input through an interface communicating with the UV sterilization apparatus.

9. A method of controlling a UV sterilization apparatus, the method comprising:
sensing a temperature in an enclosed space in which a sterilization unit is located;
when the temperature exceeds a predetermined threshold temperature, driving a motor, configured to move the sterilization unit into or out of an enclosure located in the enclosed space, to move the sterilization unit out of the enclosure located in the enclosed space; and
when the temperature becomes equal to or lower than the threshold temperature, driving the motor to move the sterilization unit back into the enclosure located in the enclosed space.

10. The method of claim 9, wherein, when moving the sterilization unit out of the enclosure located in the enclosed space, the motor is driven in a first direction, and
wherein, when moving the sterilization unit back into the enclosure located in the enclosed space, the motor is driven in a second direction, the second direction being opposite the first direction.

11. The method of claim 9, wherein the enclosed space is a vehicle, and the enclosure is an overhead console.

12. The method of claim 11, further comprising:
determining whether there is an occupant in the vehicle before the sensing of the temperature.

13. A method of controlling the UV sterilization apparatus according to claim 1, the method comprising:
receiving a sterilization request from an interface configured to communicate with the UV sterilization apparatus; and
driving the driving unit according to the received sterilization request,
wherein the sterilization unit is moved by a predetermined distance according to the received sterilization request.

14. A method of controlling the UV sterilization apparatus according to claim 4, the method comprising:
receiving a sterilization request from an interface configured to communicate with the UV sterilization apparatus; and
driving the first motor and the second motor according to the received sterilization request,
wherein the sterilization unit is moved by a predetermined distance according to the received sterilization request.

15. The method of claim 14, further comprising:
driving the first motor or the second motor to adjust an angle of the sterilization unit after the sterilization unit is moved by the predetermined distance.
